(19) United States
(12) Patent Application Publication
(10) Pub. No.: US 2023/0118283 A1
(43) Pub. Date: Apr. 20, 2023

(54) PERFORMING NEUROLOGICAL DIAGNOSTIC ASSESSMENTS

(71) Applicant: Shahnaz Miri, Washington, DC (US)

(72) Inventor: Shahnaz Miri, Washington, DC (US)

(21) Appl. No.: 18/047,447

(22) Filed: Oct. 18, 2022

Related U.S. Application Data
(60) Provisional application No. 63/262,689, filed on Oct. 18, 2021.

(51) Int. Cl. G16H 50/20; A61B 5/11; A61B 5/00

(52) U.S. Cl. CPC G16H 50/20; A61B 5/1118; A61B 5/6898; A61B 5/7267; A61B 5/1101

(57) ABSTRACT

Embodiments herein disclose computer-implemented methods, computer program products and computer systems for performing neurological diagnostic assessments. The computer-implemented method may include processors configured for receiving biometric activity data corresponding to user extremity movement from a mobile device associated with a user. Further, the computer-implemented method may include processors configured for transmitting the biometric activity data to a machine learning model. Furthermore, the computer-implemented may be configured for processing, using the machine learning model, the biometric activity data to generate first model output data corresponding to a first score. Even further, the computer-implemented method may include processors configured for determining that the first model output data corresponds to a neurological disorder classification based at least on the first score exceeding a predetermined threshold.

### CROSS-REFERENCE TO RELATED APPLICATIONS

The present application claims the benefit of the filing date of U.S. provisional application Ser. No. 63/262,689, filed, Oct. 18, 2022, the entire teachings of which are hereby incorporated herein by reference.

### BACKGROUND

The present invention relates generally to the field of diagnostic assessments, and more particularly to performing diagnostic assessments of neurological conditions.

Neurological disorders are the major cause of disability and the second cause of death globally, and the burden of these disorders continue to increase over the last decade. Neurological disorders, affecting motor and neuromuscular function, are often chronic disorders that require detailed examination by an expert neurologist to reach proper diagnosis and assess the severity of the disease. Detecting, assessing, and diagnosing neurological disorders involve various techniques and accompanying tools to assist in this endeavor.

### SUMMARY

The present invention is described in various embodiments disclosing methods, computer program products, and computer systems for performing neurological diagnostic assessments.

One embodiment of the present disclosure is a computer-implemented method for performing neurological diagnostic assessments, the computer-implemented method may include one or more processors configured for receiving biometric activity data corresponding to user extremity movement from a mobile device associated with a user.

Further, the computer-implemented method may include one or more processors configured for transmitting the biometric activity data to a machine learning model. Furthermore, the computer-implemented may include one or more processors configured for processing, using the machine learning model, the biometric activity data to generate first model output data corresponding to a first score.

Even further, the computer-implemented method may include one or more processors configured for determining that the first model output data corresponds to a neurological disorder classification based at least on the first score exceeding a predetermined threshold.

### BRIEF DESCRIPTION OF THE DRAWINGS

FIG. 1 depicts a block diagram of a system for performing neurological diagnostic assessments, in accordance with an embodiment of the present invention;

FIG. 2 depicts a body diagram of a system for performing neurological diagnostic assessments, in accordance with an embodiment of the present invention;

FIG. 3 depicts a block diagram of a model system for performing neurological diagnostic assessments, in accordance with an embodiment of the present invention;

FIG. 4 depicts a flow chart of a computer-implemented method for performing neurological diagnostic assessments, in accordance with an embodiment of the present invention;

FIG. 5 depicts a flow chart of a computer-implemented method for performing neurological diagnostic assessments, in accordance with an embodiment of the present invention;

FIG. 6 depicts muscle activity spectrograms of muscle activity at rest in the right upper extremity, in accordance with an embodiment of the present invention;

FIG. 7 depicts detection spectrograms of muscle activity in a Parkinson's Disease patient, in accordance with an embodiment of the present invention;

FIG. 8 depicts spectrograms of motor examinations in a healthy person, in accordance with an embodiment of the present invention;

FIG. 9 depicts spectrograms of motor examination in a Parkinson's Disease patient off deep brain stimulation and off medication, in accordance with an embodiment of the present invention;

FIG. 10 depicts spectrograms of motor examination in a Parkinson's Disease patient on deep brain stimulation and off medication, in accordance with an embodiment of the present invention;

FIG. 11 depicts spectrograms of motor examination in a Parkinson's Disease patient on deep brain stimulation and on medication, in accordance with an embodiment of the present invention;

FIG. 12 depicts spectrograms of neurological examination algorithms for patients with cervical dystonia, in accordance with an embodiment of the present invention;

FIG. 13 depicts spectrograms of muscle strength examination, in accordance with an embodiment of the present invention;

FIG. 14 depicts spectrograms of motor examination of muscle strength activity, in accordance with an embodiment of the present invention; and

FIG. 15 depicts a block diagram of a computing device of the distributed data processing environment of FIG. 1, in accordance with an embodiment of the present invention.

### DETAILED DESCRIPTION

The present invention addresses the problem of assessing and quantifying neurological function and motor examination, screening, diagnosing, and quantifying the severity of neurological impairment, tremor, bradykinesia, rigidity, coordination, freezing, muscle tone, muscle weakness or spasticity, in healthy people, or in patients with neurological disorders, and monitoring disease progression and disease processes. The present invention also relates to methods, algorithms, software, and application for remote assessment, telemedicine, or remote monitoring of neurological function for diagnosing and monitoring neurological diseases. The present invention also relates to clinical biomarkers for neurological disorders.

Neurological disorders are the major cause of disability and the second cause of death globally, and the burden of these disorders continue to increase over the last decade. Neurological disorders affecting motor and neuromuscular function are often chronic disorders that require detailed examination by an expert neurologist to reach proper diagnosis, assess the severity of the disease. Current measures for neurological examinations are subjective and rather dependent. There are different qualitative scales for measurement of motor examination including muscle tone, muscle strength, tremor, bradykinesia, coordination. Documentation of qualitative neurological examination performed by physicians helps long term monitoring of these patients. Disability scales, which are developed and validated for different neurological disorders, are used as biomarkers in drug development, and in data-driven therapeutic decisions in clinical practice. However, these scales are insensitive, with intraoperative variability in results, and too cumbersome to use in daily practice.

Ancillary testing provides more accurate data for assessment of motor and neuromuscular function, used for screening, diagnosing, and monitoring diseases. Traditional ancillary testing includes electromyography and nerve conduction studies, tremor studies, and gait analysis tests that require an expert clinician to perform the test in the clinical setting and an expert physician to interpret the data and discover diagnosis and underlying pathology.

Teleneurology has provided a unique opportunity for remote evaluation of patients with neurological disorders who do not have access to neurologists for evaluation or for those who are disable and have significant mobility impairment. Remote patient assessment via audio and video provides data on parts of neurological function including mental status, language, and limited motor function assessment by observation of movement in arms and legs, tremor, and gait. Remote and objective tests that can be used to support neurological examinations and disease monitoring of neuromuscular disorders and movement disorders are lacking. However, parts of the examination including muscle tone assessment, spasticity and rigidity measurement are exceptions.

Digital biomarkers are more sensitive tools compared qualitative neurological examinations and rating scales, providing objective multivariate sensor data measures that can be used in quantification of symptoms severity. Digital biomarkers can be used for self-assessment and do not rely on direct clinician assessment and provide inexpensive and accessible methods for long term monitoring of neurological disorders. Digital biomarkers can provide valid metrics to assess treatment effects and disease progression.

Smartphones and sensors are growingly used in remote patients' assessment and monitoring in different medical fields including neurology. For example, movement disorders, including Parkinson's Disease (PD), have variable features including motor and non-motor symptoms that are measured using severity scales. Unified Parkinson's Disease Severity Scale (UPDRS) has a motor scale which includes assessment of speech, facial expression, rigidity, finger tapping, hand movements, supination-pronation movements of hands, toe tapping, leg agility, tremor at rest in upper and lower extremities, postural tremor, kinetic tremor of extremities, bradykinesia, arising from chair, posture, and gait. Some components of the UPDRS motor scale have been measured using smartphone applications and sensors under research and development. However, no smartphone-derived rating score exists to comprehensively assess severity of motor symptom severity in clinical settings.

Mobile applications have been developed to measure neuromotor performance. For example, some mobile applications include Finger-Tapping measurement and different versions of Trail Making Tests. They provide preliminary assessment of motor performance and motor skills. Data obtained from the application may be used to build a normative database of individual motor skills for future translation to neurological examination. However, those mobile applications do not assess tremor, muscle tone, or rigidity, which are critical components of motor examination.

Other example mobile applications may include tablet versions of the Spiral Drawing Test for PD patients improve the likelihood of detecting tremors. Other mobile applications employ a combination of tapping tests (to measure speed and accuracy of tapping) and spiral drawing tests for patients with PD.

Further, other mobile applications perform assessments with additional motor exam components including voice, posture, gait (walk test), finger tapping, and response time (press and hold the on-screen button as instructed by the application) as an attempt to develop and validate a feasible smartphone-derived severity score for PD with objective measure of motor symptoms. For example, those attempts generated a mobile Parkinson disease score mPDS) using a machine-learning—based approach analysis of different test components, scaled from 0 to 100 (higher scores reflect more severe disease). Other mobile applications record gait movements during walking using smartphone sensors and have been validated in PD gait assessment.

Furthermore, neurological disorders may be assessed for hand and leg tremor, gait, and turning using the smartphone 3D accelerometer and a cloud data processing. For example, this assessment instructs the user to attach the smartphone to the back of their hands and leg while walking, instead of using external sensors to measure motion data in PD patients. However, these types of assessments do not provide information of bradykinesia, rigidity, or muscle tone.

Electromyography (EMG) is a traditional method for assessment of neuromuscular function, using surface electrode recording and needle recording of muscle electrical activity. Myoelectrical activity of different muscle groups can provide information on muscle tone, neuromuscular function, and muscle strength. Surface electromyography (EMG) and muscle sonography are sensitive tools for detection of muscle fasciculations. EMG should be performed and interpreted by an expert in the clinical setting to achieve adequate diagnostic reliability. Previous studies suggest muscle ultrasonography is more convenient and reliable than surface EMG in detecting neuromuscular abnormalities. With the advancement of technology, EMG sensors are being developed for remote assessment and monitoring of muscle activities. The latest wearable technologies include multiple sensors attached to different parts of patients' bodies to evaluate muscle contractions. Electrophysiological studies help with classification of tremors in movement disorders. Surface EMG data can be used in assessment of PD symptoms, mainly assessment of resting tremor. Online platforms have been developed for tremor analysis by comparing the activity recorded on the accelerometer and EMG data obtained from extensor carpi radialis and flexor carpi radialis muscles. For example, wrist-worn wearable neuromodulation devices record tremor activity from the wrist and provides neuromodulation for treatment of essential tremor.

Innovative mobile and wearable technologies might streamline the execution of clinical trials. Wearable sensors allow remote patient monitoring and may provide insight into a disease status and response to medications. However, patients' adherence to using wearables feasibility, accessibility, and cost-effectiveness of such devices are not clearly known. Although numerous commercial digital devices are available, each have limited features and there is no comprehensive motor assessment which includes muscle tone, rigidity assessment and other neuromuscular function.

The present invention relates to computer-implemented methods and computer systems for assessing and quantifying neurological function and motor examination, screening, diagnosing, and quantifying the severity of neurological impairment, tremor, bradykinesia, rigidity, coordination, freezing, muscle tone, muscle weakness or spasticity, in healthy people, or in patients with neurological disorders, and monitoring disease progression and disease processes. Embodiments of the present invention also relate to computer-implemented methods, algorithms, computer software, and applications for remote assessment, telemedicine, or remote monitoring of neurological function for diagnosing and monitoring neurological diseases. Embodiments of the present invention also relate to clinical biomarkers for neurological disorders.

In an embodiment, the computer-implemented method for performing neurological diagnostic assessments may be configured for motor examination and neuromuscular function testing. For example, the computer-implemented method may include one or more processors configured to receive muscle activity data corresponding to one or more of a muscle abnormality (e.g., tremor, postural tremor), muscle acoustics (e.g., muscle baseline tone, muscle tone and rigidity), normal muscle activity (e.g., finger tapping, hand movements), muscle testing activity (e.g., rapid alternating hand movements, foot movements, leg agility movements) detected in one or more of the left and right extremities, the upper and lower extremities of a user body, by an audio sensor (e.g., microphone) of a computing device (e.g., mobile device, smartphone). The muscle activity data may be received while the user body is at rest or while the user body is performing some muscular activity (e.g., described above). Once the muscle activity data is received, the one or more processors may be configured to perform analysis based on tremor frequency, amplitude, and regularity.

In an embodiment, the one or more processors may be configured to receive muscle activity data corresponding to a postural tremor detected in one or more of the left and right upper extremities while an arm is outstretched or the lower extremities while a leg is outstretched, wherein a diagnostic analysis may be performed based on tremor frequency, amplitude, and regularity.

In an embodiment, the one or more processors may be configured to receive muscle activity data corresponding to muscle baseline tone detected in one or more of the left and right, upper and lower extremities by a microphone sensor of a smartphone while the user body is at rest, wherein a diagnostic analysis may be performed based on muscle contraction sound (e.g., intensity, frequency).

In an embodiment, the one or more processors may be configured to receive muscle activity data corresponding to muscle tone and rigidity in one or more of the left and right extremities by a microphone sensor of a smartphone during upper extremity flexion and elbow extension, wherein a diagnostic analysis may be performed based on muscle contraction sound (e.g., intensity, frequency).

In an embodiment, the one or more processors may be configured to receive muscle activity data corresponding to finger tapping in one or more of the left and right hand by a microphone sensor of a smartphone during finger tapping, wherein a diagnostic analysis may be performed based on muscle contraction sound (e.g., intensity, frequency).

In an embodiment, the one or more processors may be configured to receive muscle activity data corresponding to hand movements in one or more of the left and right upper extremities (e.g., arms, hands) by a microphone sensor of a smartphone during hand movements during hand opening and hand closing, wherein a diagnostic analysis may be performed based on muscle contraction sound (e.g., intensity, frequency) and hand movement (e.g., speed, rhythm).

In an embodiment, the one or more processors may be configured to receive muscle activity data corresponding to rapid alternating hand movements in one or more of the left and right upper extremities by a microphone sensor of a smartphone during hand supination-pronation, wherein a diagnostic analysis may be performed based on muscle contraction sound (e.g., intensity, frequency) and hand movement (e.g., speed, rhythm).

In an embodiment, the one or more processors may be configured to receive muscle activity data corresponding to foot movements in one or more of the left and right lower extremities by a microphone sensor of a smartphone during foot tapping, wherein a diagnostic analysis may be performed based on muscle contraction sound (e.g., intensity, frequency), and foot movement (e.g., speed of foot tapping, rhythm).

In an embodiment, the one or more processors may be configured to receive muscle activity data corresponding to leg agility movements in one or more of the left and right lower extremities by a microphone sensor of a smartphone during leg agility tasks, wherein a diagnostic analysis may be performed based on muscle contraction sound (e.g., intensity, frequency), foot movement (e.g., speed of foot tapping, rhythm), and leg agility amplitude.

In an embodiment, the computer-implemented method for performing neurological diagnostic assessments may include one or more processors configured for receiving biometric activity data corresponding to user extremity movement from a mobile device associated with a user, transmitting the biometric activity data to a machine learning model. Additionally, the machine learning model may receive training data corresponding to baseline muscle activity data for a user and abnormal muscle activity data corresponding to a neurological disorder clinical diagnosis for a user, wherein the trained machine learning model may generate model output data corresponding to one or more classifications of a patient's neurological status. For example, the trained machine learning model may be configured to classify neurological disorders more accurately than the machine learning model based on a comparison between the second score and the first score exceeding a threshold.

In an embodiment, the one or more processors may be configured for presenting user prompts instructing the user to perform each muscle activity task in a specific order and to initiate muscle activity recording for a specific time duration (e.g., 1 second, 5 seconds, 10 seconds, or more time as needed). Further, the one or more processors may be configured to present subsequent user prompts after muscle activity tasks are completed, wherein the subsequent user prompts may include instructions to the user about how to perform the next task. Upon the user completing all the specified tasks, the one or more processors may be configured to transmit data corresponding to the recorded muscle activity data to a network for analytics using machine learning algorithms. For example, a combination of the recorded muscle activity data may be used for assessing patient movement disorders by comparing the recorded muscle activity data to baseline data corresponding to healthy demographics consistent controls.

In yet another embodiment, the computer-implemented method for performing neurological diagnostic assessments may include one or more processors configured for muscle strength testing. For example, the one or more processors may be configured for analyzing muscle activity at rest, and in outstretched arms and legs compared to muscle activity while holding weights (e.g., 2 lbs., 4 lbs.). Further, the one or more processors may be configured for detecting patterns of muscle fatigue related to neuromuscular disorders including myasthenia gravis, detecting patterns of muscle fasciculation related to neuromuscular disorders including amyotrophic lateral sclerosis, detecting focal muscle weakness in upper or lower extremities. Furthermore, the one or more processors may be configured for screening, diagnosing, and monitoring neurological disorders with focal motor deficit including neurovascular disorders (stroke), multiple sclerosis and other neurodegenerative and autoimmune disorders of the central nervous system. For example, the one or more processors may be configured for detecting focal muscle spasticity and dystonia based on measurement of muscle activity recording at rest and with action in upper and lower extremities.

In yet another embodiment, the computer-implemented method for performing neurological diagnostic assessments may include one or more processors for motor examination and neuromuscular function, wherein the model output data may be processed using a scoring system to classify different neurological disorders as described above herein.

The present invention will now be described in detail with reference to the Figures.

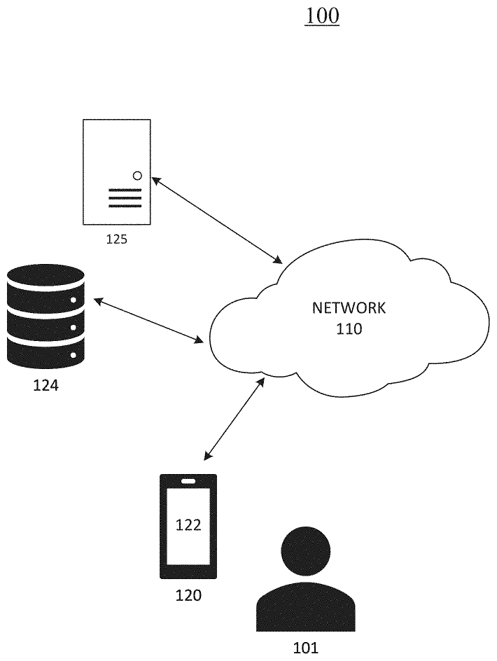

FIG. 1 depicts a block diagram of a distributed data processing environment 100 for performing neurological diagnostic assessments, in accordance with an embodiment of the present invention. FIG. 1 provides only an illustration of one embodiment of the present invention and does not imply any limitations with regard to the environments in which different embodiments may be implemented. In the depicted embodiment, distributed data processing environment 100 includes computing device 120 (with user interface 122, server 125, and database 124 interconnected over network 110. Network 110 operates as a computing network that can be, for example, a local area network (LAN), a wide area network (WAN), or a combination of the two, and can include wired, wireless, or fiber optic connections. In general, network 110 can be any combination of connections and protocols that will support communications between computing device 120, server 125, and database 124. Distributed data processing environment 100 may also include additional servers, computers, sensors, or other devices not shown.

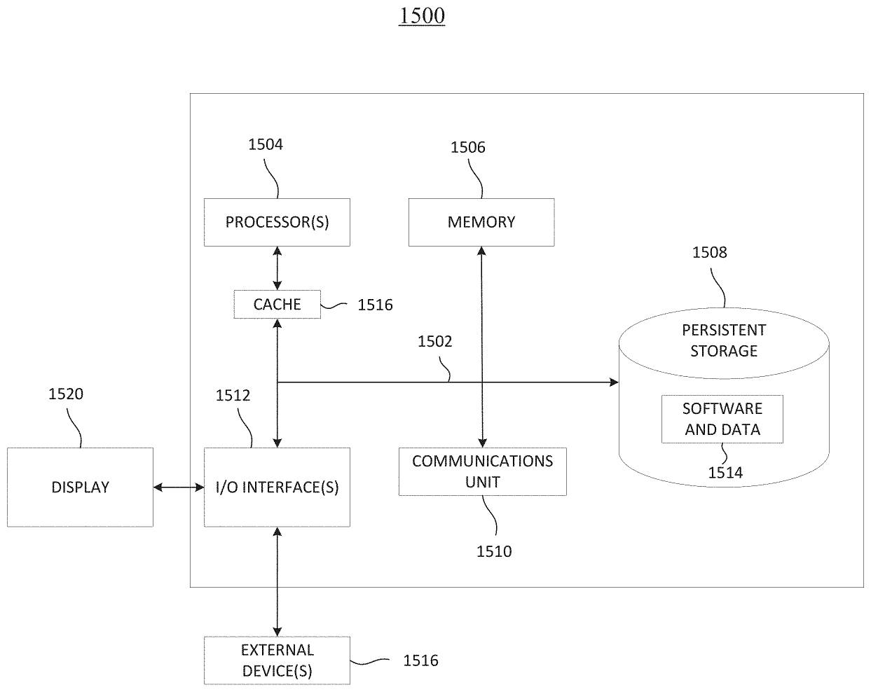

Computing device 120 operates to execute at least a part of a computer program for performing neurological diagnostic assessments. In an embodiment, computing device 120 may be communicatively coupled with a microphone (not shown) or the microphone may be one of computing device 120 components. Computing device 120 be configured to send and/or receive data from network 110. In some embodiments, computing device 120 may be a management server, a web server, or any other electronic device or computing system capable of receiving and sending data. In some embodiments, computing device 120 may be a laptop computer, tablet computer, netbook computer, personal computer (PC), a desktop computer, a smart phone, or any programmable electronic device capable of communicating with database(s) 124, server(s) 125 via network 110. Computing device 120 may include components as described in further detail in FIG. 15.

Computing device 120 may also be configured to receive, store, and process audio data received by the microphone. For example, computing device 120 may be communicatively coupled to the microphone and receive, via a communications link, audio data corresponding to sounds captured by the microphone. Computing device 120 may be configured to store the audio data in memory of computing device 120 or transmit the audio data to database 124 or server 125 via network 110. The audio data may be processed by one or more processors of computing device 120 or by one or more processors associated with server(s) 125 in a cloud computing network.

Database 124 operates as a repository for data flowing to and from network 110. Examples of data include user data, device data, network data, and data corresponding to audio detected by the microphone. A database is an organized collection of data. Database 124 can be implemented with any type of storage device capable of storing data and configuration files that can be accessed and utilized by computing device 120, such as a database server, a hard disk drive, or a flash memory. In an embodiment, database 124 is accessed by computing device 120 to store data corresponding to audio captured by the microphone. In another embodiment, database 124 is accessed by computing device 120 to access user data, device data, network data, and data corresponding to audio captured by the microphone. In another embodiment, database 124 may reside elsewhere within distributed network environment 100 provided database 124 has access to network 110.

Server 125 can be a standalone computing device, a management server, a web server, or any other electronic device or computing system capable of receiving, sending, and processing data and capable of communicating with computing device 120 via network 110. In other embodiments, server 125 represents a server computing system utilizing multiple computers as a server system, such as a cloud computing environment. In yet other embodiments, server 125 represents a computing system utilizing clustered computers and components (e.g., database server computers, application server computers, etc.) that act as a single pool of seamless resources when accessed within distributed data processing environment 100. Server 125 may include components as described in further detail in FIG. 15.

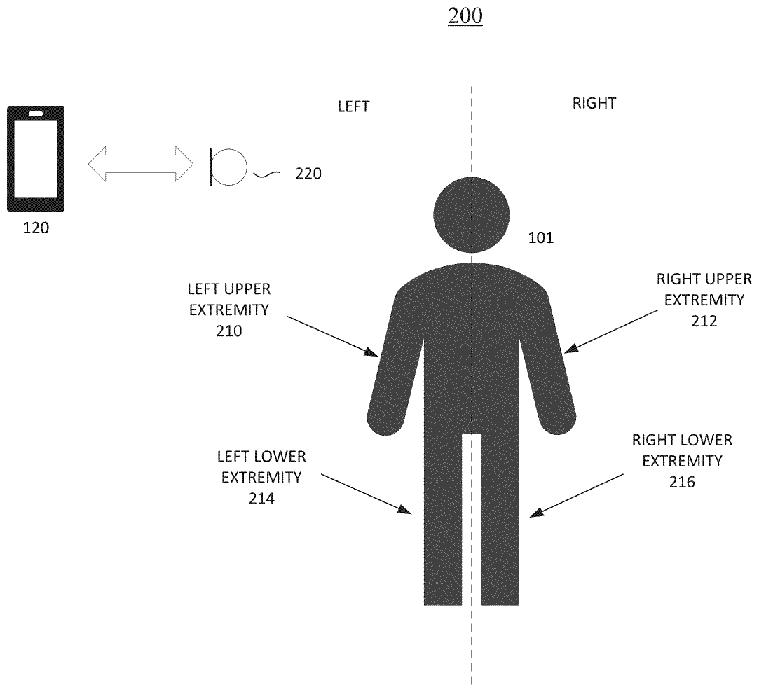

FIG. 2 depicts a body diagram of a system 200 for performing neurological diagnostic assessments, in accordance with an embodiment of the present invention.

In an embodiment, system 200 may include one or more processors configured to execute a computer program (e.g., smartphone application) to generate a user-interface on a display of computing device 120, wherein the computer program may be configured to collect audio data of muscle activity recorded by placement of a microphone 220 sensor (i.e., a sensory embedded in mobile device 120 or an external microphone sensor) over the skin of the body of user 101. For example, the body of user 101 may be divided into left and right sides of upper and lower extremities, characterized as left upper extremity 210 (LUE), right upper extremity 212 (RUE), left lower extremity 214 (LLE), and right lower extremity 216 (RLE). Further, audio data of muscle activity may be recorded by microphone 220 over the skin of the body of user 101 proximate to other body parts or muscle groups (e.g., head, neck, back, trunk), wherein instructions may be presented to user 101 to capture the audio data while at rest or while activating other muscle groups or body parts (e.g., knee extension, elbow extension, finger tapping, rapid alternating hand movements, foot tapping, leg agility, holding weights).

In an embodiment, the one or more processors may be configured to generate a user interface on mobile device 120 to label and/or tag data as the data is received at mobile device 120 or data made available at mobile device 120, wherein the label and/or tag data may be based on medial and neurological guidelines with various protocols for combination of tests in assessment of different health conditions.

In an embodiment, the one or more processors may be configured to execute a battery of tests locally or virtual (remotely), wherein the data accumulated during the tests may be transferred to a cloud server to perform additional processing (e.g., automatic quantification using machine learning, pattern recognition, audio analysis).

In an embodiment, the one or more processors may be configured to generate a real-time result page with a detailed analysis of different test components, scoring system, and comparison of patients with normative data, or longitudinal comparison of one subject with multiple recordings.

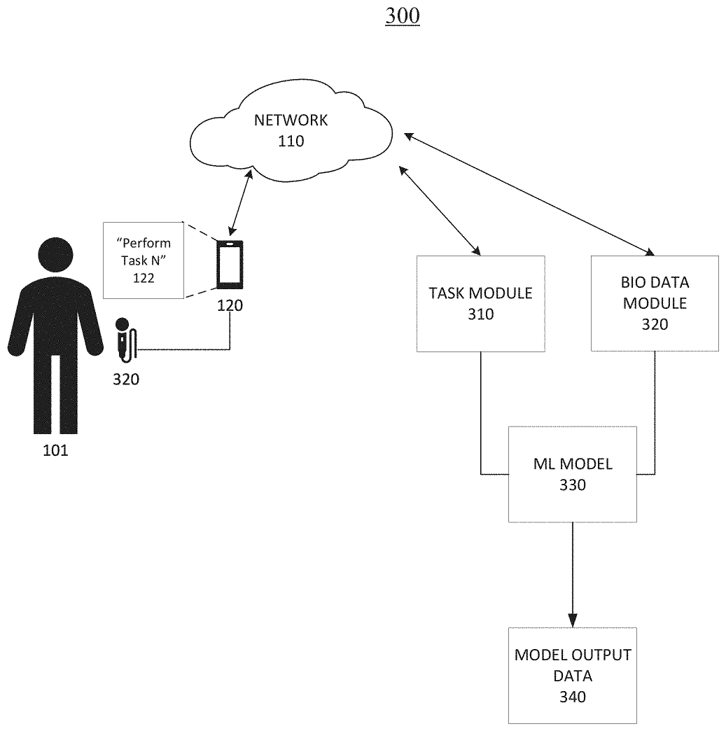

FIG. 3 depicts a block diagram of a model system 300 for performing neurological diagnostic assessments, in accordance with an embodiment of the present invention.

In an embodiment, model system 300 may include one or more processors configured for assessing neuro-muscular function based on motor examination. For example, model system 300 may include task module 310 configured to generate tasks for presentation to the user to perform (e.g., perform Task N 122), bio data module 320 configured to generate biometric activity data corresponding to muscle activity data recorded by mobile device 120, machine learning model 330 configured to process the biometric activity data to generate model output data 340 corresponding to a neurological disorder classification.

While the foregoing describes, and FIG. 3 illustrates, implementation of model 330 comprising machine learning (ML) model 330, the present disclosure is not limited thereto. In at least some embodiments, model 330 may implement a trained component or trained model configured to perform the processes described above with respect to the model 330. The trained component may include one or more machine learning models, including but not limited to, one or more classifiers, one or more neural networks, one or more probabilistic graphs, one or more decision trees, and others. In other embodiments, the trained component may include a rules-based engine, one or more statistical-based algorithms, one or more mapping functions or other types of functions/algorithms to determine whether a natural language input is a complex or non-complex natural language input. In some embodiments, the trained component may be configured to perform binary classification, where the natural language input may be classified into one of two classes/categories. In some embodiments, the trained component may be configured to perform multiclass or multinomial classification, where the natural language input may be classified into one of three or more classes/categories. In some embodiments, the trained component may be configured to perform multi-label classification, where the natural language input may be associated with more than one class/category.

Various machine learning techniques may be used to train and operate trained components to perform various processes described herein. Models may be trained and operated according to various machine learning techniques. Such techniques may include, for example, neural networks (such as deep neural networks and/or recurrent neural networks), inference engines, trained classifiers, etc. Examples of trained classifiers include Support Vector Machines (SVMs), neural networks, decision trees, AdaBoost (short for “Adaptive Boosting”) combined with decision trees, and random forests. Focusing on SVM as an example, SVM is a supervised learning model with associated learning algorithms that analyze data and recognize patterns in the data, and which are commonly used for classification and regression analysis. Given a set of training examples, each marked as belonging to one of two categories, an SVM training algorithm builds a model that assigns new examples into one category or the other, making it a non-probabilistic binary linear classifier. More complex SVM models may be built with the training set identifying more than two categories, with the SVM determining which category is most similar to input data. An SVM model may be mapped so that the examples of the separate categories are divided by clear gaps. New examples are then mapped into that same space and predicted to belong to a category based on which side of the gaps they fall on. Classifiers may issue a “score” indicating which category the data most closely matches. The score may provide an indication of how closely the data matches the category.

In order to apply the machine learning techniques, the machine learning processes themselves need to be trained. Training a machine learning component requires establishing a “ground truth” for the training examples. In machine learning, the term “ground truth” refers to the accuracy of a training set's classification for supervised learning techniques. Various techniques may be used to train the models including backpropagation, statistical learning, supervised learning, semi-supervised learning, stochastic learning, or other known techniques.

In an embodiment, model system 300 may include one or more processors configured for executing a recognition method computer program based on recorded surface muscular sounds using microphone 320. For example, muscle sound recordings may be classified with a two-phase algorithm (e.g., signal analysis and pattern classifier using machine learning algorithms), wherein each audio recording data may be passed to the appropriate analytic model in an auto-scaling server environment. Further, for example, each analytic model may be designed to accept an audio file and return the results for that test, wherein results from each process of the test may be stored in a database.

In an embodiment, model system 300 may include one or more processors configured to perform Audio Spectral Analysis, wherein the biometric activity data may be processed to generate spectrograms to identify features corresponding to spectral power spectral density (PSD), voice intensity, pitch, formants, and linear predictive coding (LPC). For example, a spectrogram is a visual representation of the short-time spectrum of frequencies along the time axis. Further, for example, PSD is the measure of the signal's power/energy at each frequency slot, voice intensity is the volume of the sound, and pitch is the glottal vibration frequency.

In an embodiment, model system 300 may include one or more processors configured to execute a multimodal approach to include the subject muscle activity and a combination of accelerometer data. For example, the audio data from muscle activity and accelerometer data of motion are used together to aid the assessment of motor function and neurological examination. Further, additional audio data including speech data may be used for assessment of speech and language, dysarthria and hypophonia.

In an embodiment, model system 300 may include one or more processors configured to compute different components of neurological function and may provide quantitative analysis and qualitative results based on the standardized scales, including UPDRS motor score. For example, total score is computed by weighing and summing the individual scores in each task, wherein the weights may be learned from the data using machine learning.

In an embodiment, the one or more processors may be configured to generate a one-page report output of individual case analysis after each test with explanation that may include three sections: (a) Summary Section: a simple numerical score (e.g., 0 to 4) for each test component; (b) Detail Section: detailed quantification of audio analysis and results and comparison with normal range; and (c) Trend Section: a histogram of test scores to help patients assess changes in their score over time. Furthermore, additional interpretation of individual cases compared to normal range may be performed via telemedicine visits. Additionally, the one or more processors may be configured to generate an export or email option so the patient can easily share the data with their doctor.

In an embodiment, model system 300 may include one or more processors configured to automatically perform the tests with the aid of machine learning technologies (e.g., Voice Activity Detection “VAD”, and Audio Spectral Analyses). Specifically, VAD events include at rest muscle tone, tremors, muscle fibrillation, muscle contraction pattern while performing different motor tasks. For example, a sample of 100 control cases may be used as a control database for each task to produce normal range values, wherein the results from all other cases may be reported compared to normal values.

In an embodiment, model system 300 may include one or more processors configured to use VAD to recognize muscle tone, spasticity, tremor, rigidity, fibrillation potentials, fibrillations, muscle weakness, bradykinesia, freezing of movements, ataxia, and other abnormal neurological findings.

In an embodiment, model system 300 may include one or more processors configured to use VAD to analyze neurological findings recorded from different muscle groups in the body, to recognize asymmetry of neurological function in the left versus right side of the body, and to evaluate and classify different neurological conditions based on the recorded data and pre-developed database.

In an embodiment, model system 300 may include one or more processors configured to evaluate the reliability of the voice-based and image-based physical examination data. For example, the reliability may be evaluated using accelerometer data input over time scale which may confirm frequency, amplitude, and velocity of motor tasks. Therefore, the accelerometer data may be used for assisting screening for errors or unreliable task performance or collected data. Furthermore, comparison of recorded data with normal database can reveal unreliable data patterns and the validity and/or reliability of the test may be examined separately in various clinical studies by comparing the test and the test results with a standard physician scoring system.

In an embodiment, model system 300 may include one or more processors configured to collect metadata through voice on the subject's age, gender, condition, medical history, and activities before testing, wherein embodiments described herein may be extended for virtual physical examinations and neurological self-assessment for healthy people or those with underlying conditions.

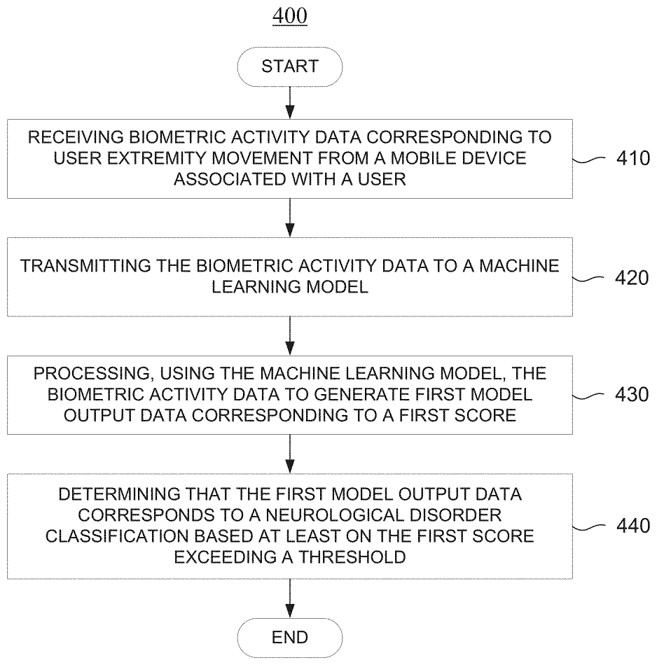

FIG. 4 depicts a flow chart of computer-implemented method 400 for performing neurological diagnostic assessments, in accordance with an embodiment of the present invention.

In an embodiment, computer-implemented method 400 may include one or more processors configured for receiving 410 biometric activity data corresponding to user extremity movement from a mobile device associated with a user.

In an embodiment, computer-implemented method 400 may include one or more processors configured for transmitting 420 the biometric activity data to a machine learning model.

In an embodiment, computer-implemented method 400 may include one or more processors configured for processing 430, using the machine learning model, the biometric activity data to generate first model output data corresponding to a first score.

In an embodiment, computer-implemented method 400 may include one or more processors configured for determining a neuromuscular fitness score based on the biometric activity data by evaluating motor unit activation parameters in athletes and non-athletes.

In an embodiment, computer-implemented method 400 may include one or more processors configured for determining neuromuscular responses, neurological deficit progression or improvement by comparing model output data for the same subject user over time.

In an embodiment, computer-implemented method 400 may include one or more processors configured for determining 440 that the first model output data corresponds to a neurological disorder classification based at least on the first score exceeding a threshold.

In an embodiment, computer-implemented method 400 may include one or more processors configured for presenting instructions via a user interface of the mobile device instructing the user to perform one or more tasks.

In an embodiment, responsive to presenting the instructions, computer-implemented method 400 may include one or more processors configured for receiving audio data from a biometric sensor of the mobile device contemporaneous with the one or more tasks.

In an embodiment, the biometric activity data may be based at least on the audio data received contemporaneously with the user performing the one or more tasks.

In an embodiment, the one or more tasks may include positioning the biometric sensor of the mobile device at a specific location about the body of the user to capture the audio data while the user is at rest or performing the one or more tasks.

In an embodiment, the specific location may be selected from a group consisting of left upper extremity, right upper extremity, left lower extremity, right lower extremity, left hand, right hand, left foot, right foot, and other muscle groups including neck in the case of cervical dystonia.

In an embodiment, computer-implemented method 400 may include one or more processors configured for receiving at the machine learning model, training data corresponding to normal neurological disorder profiles and abnormal neurological disorder profiles.

In an embodiment, computer-implemented method 400 may include one or more processors configured for processing at the machine learning model, the training data to configure a trained machine learning model.

In an embodiment, computer-implemented method 400 may include one or more processors configured for transmitting the biometric activity data to the trained machine learning model and processing using the trained machine learning model, the biometric activity data to generate second model output data corresponding to a second score.

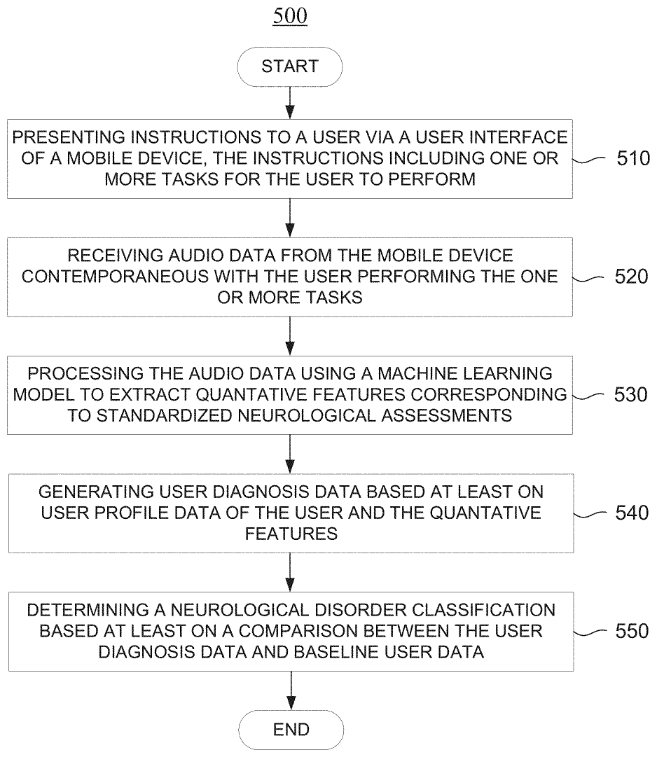

FIG. 5 depicts a flow chart of computer-implemented method 500 for performing neurological diagnostic assessments, in accordance with an embodiment of the present invention.

In an embodiment, computer-implemented method 500 may include one or more processors configured for presenting 510 instructions to a user via a user interface of a mobile device, the instructions including one or more tasks for the user to perform.

In an embodiment, computer-implemented method 500 may include one or more processors configured for receiving 520 audio data from the mobile device contemporaneous with the user performing the one or more tasks.

In an embodiment, computer-implemented method 500 may include one or more processors configured for processing 530 the audio data using a machine learning model to extract quantitative features corresponding to standardized neurological assessments.

In an embodiment, computer-implemented method 500 may include one or more processors configured for generating 540 user diagnosis data based at least on user profile data of the user and the quantitative features.

In an embodiment, computer-implemented method 500 may include one or more processors configured for determining 550 a neurological disorder classification based at least on a comparison between the user diagnosis data and baseline user data.

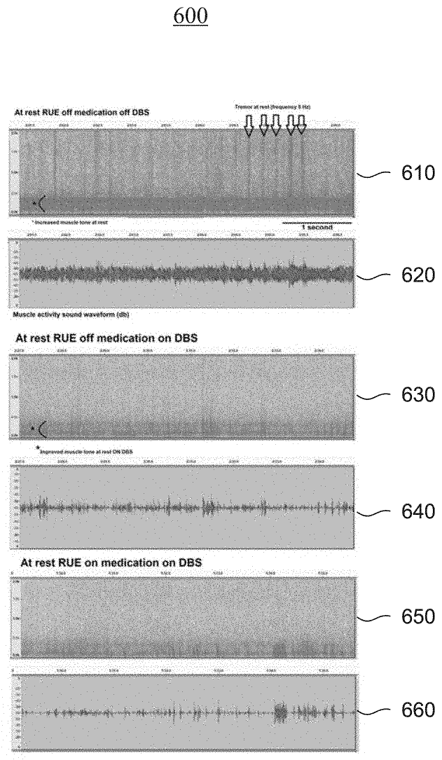

FIG. 6 depicts muscle activity spectrograms 600 of muscle activity at rest in the right upper extremity, in accordance with an embodiment of the present invention.

In an embodiment, FIG. 6 illustrates spectrograms of muscle activity at rest in the right upper extremity, demonstrating increased muscle tone and high level of muscle activity at rest with 5 Hz tremor, which improves after deep brain stimulation (DBS) and taking Parkinson medication, according to one embodiment of the present invention.

For example, at rest muscle tone spectrogram 610 illustrates a detected tremor at rest with frequency of 5 Hz (in 1 second recording) typical for Parkinson's disease, wherein the detected tremor is evidence of abnormal muscle activity (increased tone/ rigidity) in the right upper extremity (RUE) represented as an abnormal sound wave at that point in time when the audio was recorded while the patient was off medication and off DB S (untreated patient). Further, at rest muscle tone spectrogram 610 illustrates an ever-present increased muscle tone while the patient is at rest, indicating abnormal muscle activity represented at the bottom portion of the spectrogram. Even further as an example, at rest muscle activity sound waveform 620 illustrates abnormal muscle activity in the form of an ever-present detectable sound intensity while the patient is at rest. It should be noted that the tremor at rest in at rest muscle tone spectrogram 610 is consistent with a contemporaneous increased muscle sound intensity (indicating abnormal neuromuscular unit recruitment) in at rest muscle activity sound waveform 620.

In an embodiment, at rest muscle tone spectrogram 630 illustrates evidence of improved muscle activity in the right upper extremity (RUE) represented as improved muscle tone at rest on DBS at that point in time when the audio was recorded while the patient was off medication and on DBS. Further, at rest muscle tone spectrogram 630 illustrates an ever-present improved muscle tone while the patient is at rest, indicating improved muscle activity represented at the bottom portion of the spectrogram, without any evidence of tremor at rest. Even further as an example, at rest muscle activity sound waveform 640 illustrates improved muscle activity in the form of a decreased ever-present detectable sound intensity while the patient is at rest, as compared to at rest muscle activity sound waveform 620 (untreated patient status).

In an embodiment, a similar phenomenon may be observed between at rest muscle tone spectrogram 650 and at rest muscle activity sound waveform 660 for a patient at rest who treated with medication and on DBS.

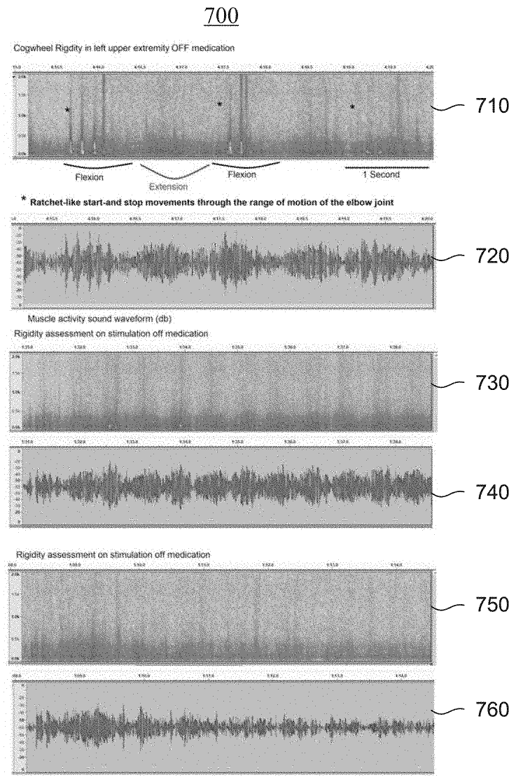

FIG. 7 depicts detection spectrograms 700 of muscle activity in a Parkinson's Disease patient, in accordance with an example embodiment of the present invention.

FIG. 7 illustrates detection of cogwheel rigidity in the spectrogram (e.g., 710, 730, 750) and corresponding waveforms (e.g., 720, 740, 760) of muscle activity from patients with Parkinson's disease. Cogwheel Rigidity is detectable when the patient is untreated (off medication, off DBS) in the spectrogram 710 and waveform 720 while performing flexion extension of extremities. In an embodiment, cogwheel rigidity improves after turning on deep brain stimulation and taking medications (730, 740, 750, 760), showing the technical benefits of embodiments described herein as a biomarker, according to an example embodiment of the present invention.

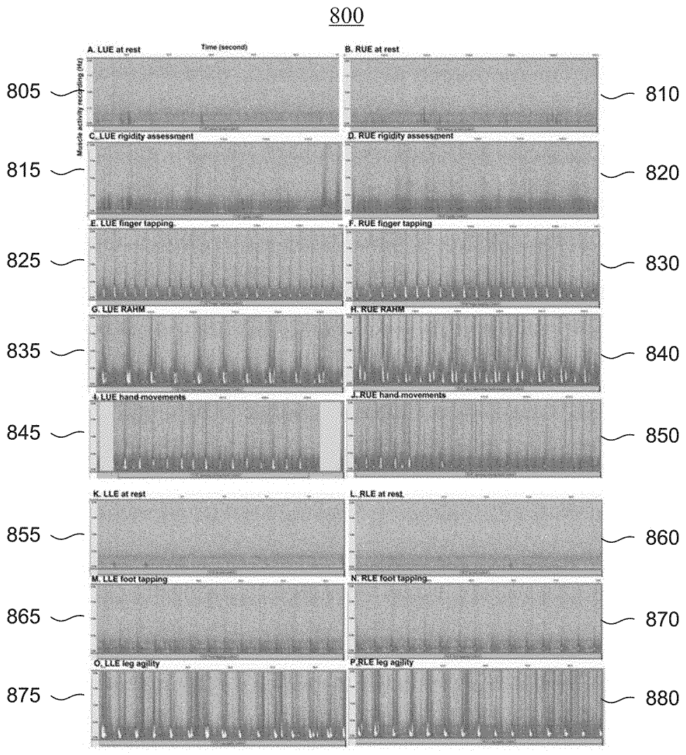

FIG. 8 depicts muscle activity spectrograms 800 while performing motor examinations in a healthy person, in accordance with an example embodiment of the present invention.

FIG. 8 illustrates muscle activity spectrograms 800 of motor examinations in a healthy person who has performed different tasks in the test. At rest muscle tone (e.g., LUE 805, RUE 810) rigidity assessment while performing flexion-extension of extremities (e.g., LUE 815, RUE 820), finger tapping (e.g., LUE 825, RUE 830), rapid alternating hand movements (RAHM) (e.g., LUE 835, RUE 840), and hand movements (e.g., opening and closing hands) (e.g., LUE 845, RUE 850) in the right and left upper extremities. Additionally, muscle tone in lower extremities at rest (e.g., LLE 855, RLE 860), while foot tapping (e.g., LLE 865, RLE 870) and performing leg agility (heel tapping) (e.g., LLE 875, RLE 880) tests in both legs are demonstrated, according to example embodiments of the present invention.

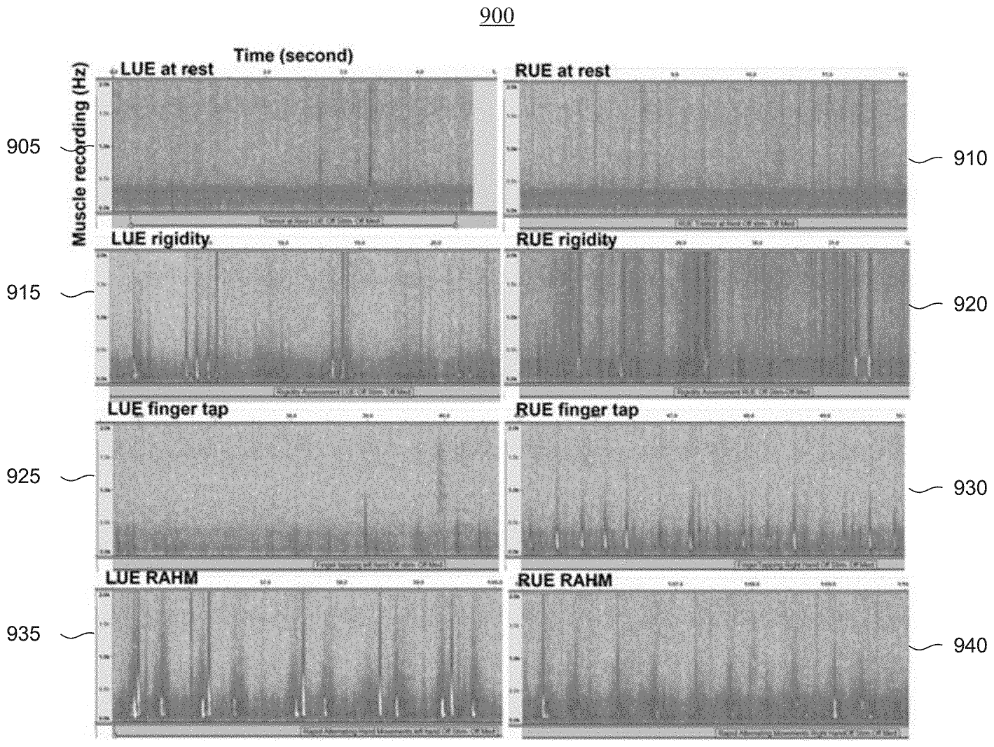

FIG. 9 depicts spectrograms 900 of motor examination in a Parkinson's Disease (PD) patient off deep brain stimulation and off medication, in accordance with an example embodiment of the present invention.

FIG. 9 illustrates spectrograms 900 of motor examination in a PD patient off deep brain stimulation and off medication. In an embodiment, at rest muscle tone (e.g., LUE 905, RUE 910) is increased in both upper extremities compared to healthy control. Cogwheel rigidity (e.g., LUE 915, RUE 920) is notable in bilateral upper extremities and freezing (irregular and arrhythmic muscle activity) in finger tapping (e.g., LUE 925, RUE 930) in left and bradykinesia (lower frequency of movements) in both hands during the finger tapping test is demonstrated. Rapid alternating hand movements (e.g., LUE 935, RUE 940) with different amplitude and variable rhythm are detectable, according to an example embodiment of the present invention. Asymmetry of rigidity and bradykinesia is evident in this patient, symptoms are worse in the left side of the body.

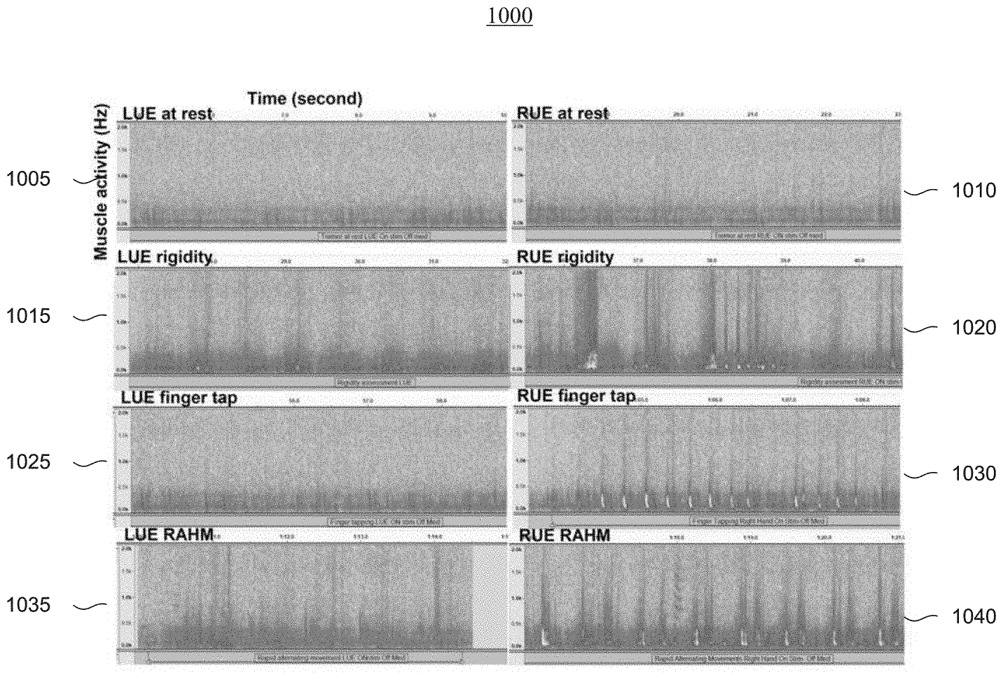

FIG. 10 depicts spectrograms 1000 of motor examination in a Parkinson's Disease patient on deep brain stimulation and off medication, in accordance with an example embodiment of the present invention.

FIG. 10 illustrates spectrograms 1000 of an embodiment of motor examination in the PD patient on deep brain stimulation off medication. In an embodiment, the illustrations reveal changes in neurological examination after turning on DBS, which includes decreased muscle tone at rest (e.g., LUE 1005, RUE 1010), decreased rigidity (e.g., LUE 1015, RUE 1020) in both upper extremities, improvement of finger tapping (e.g., LUE 1025, RUE 1030) and freezing, according to an embodiment of the present invention. Rapid alternating hand movements (e.g., LUE 1035, RUE 1040) with different amplitude and variable rhythm are detectable, according to an example embodiment of the present invention.

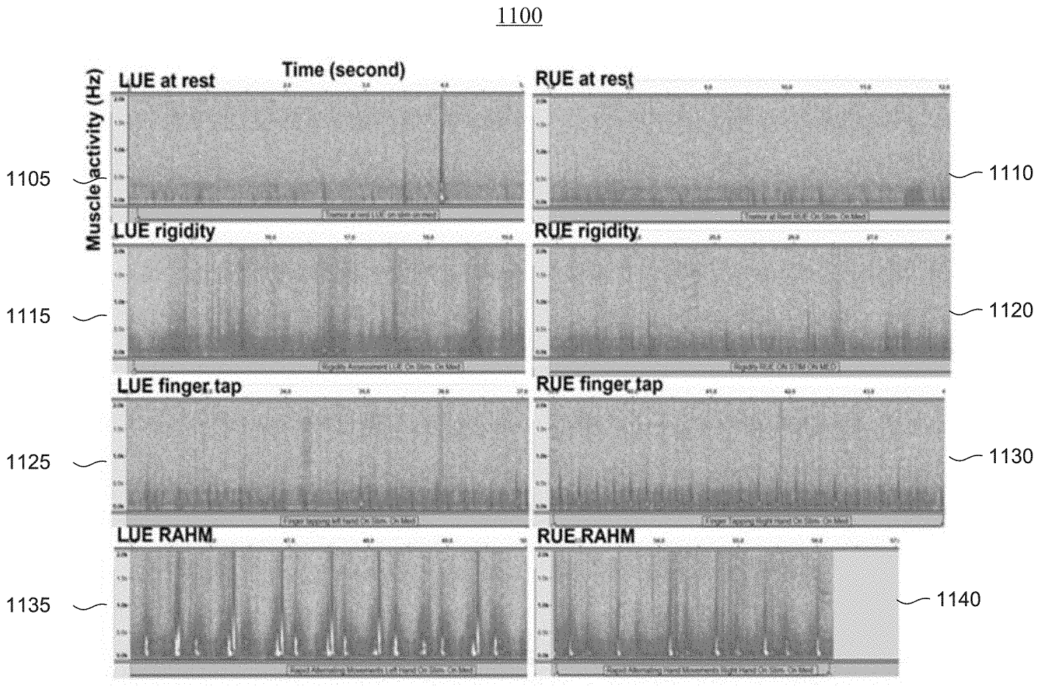

FIG. 11 depicts spectrograms 1100 of motor examination in a Parkinson's Disease patient on deep brain stimulation and on medication, in accordance with an example embodiment of the present invention.

FIG. 11 illustrates a diagram of spectrograms 1100 of an example embodiment of motor examination in the PD patient on deep brain stimulation and on medication. In an embodiment, improvement of muscle tone and tremor at rest (e.g., LUE 1105, RUE 1110), improvement of rigidity (e.g., LUE 1115, RUE 1120), finger tapping (e.g., LUE 1125, RUE 1130), bradykinesia, and rapid alternating hand movements (RAHM) (e.g., LUE 1135, RUE 1140) are demonstrated based on the test results, according to an example embodiment of the present invention.

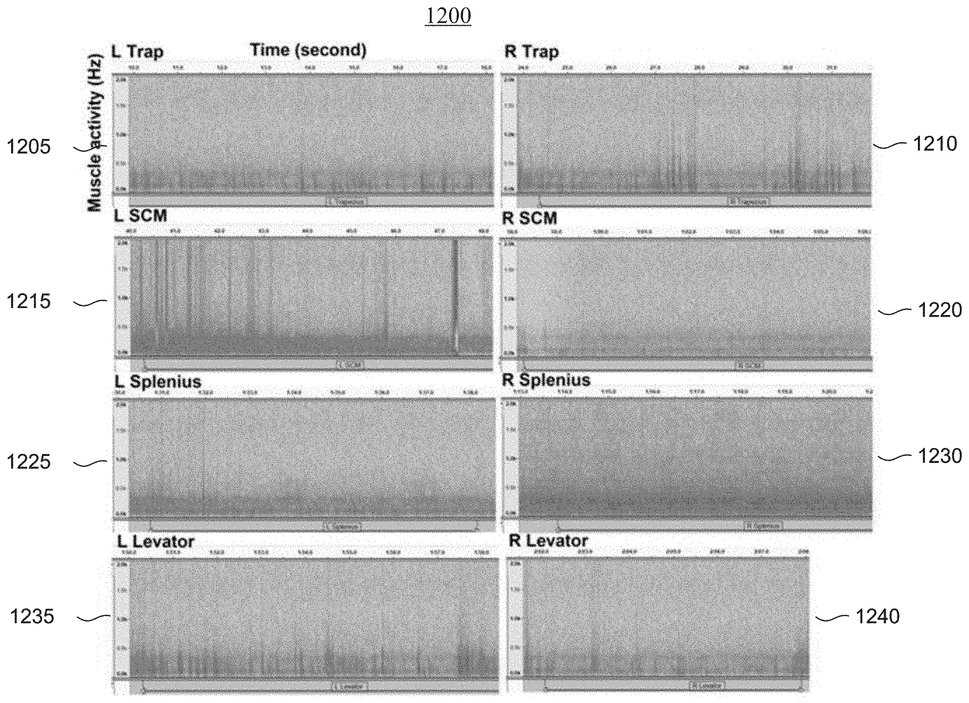

FIG. 12 depicts spectrograms 1200 of neurological examination algorithms for patients with cervical dystonia, in accordance with an embodiment of the present invention.

FIG. 12 illustrates a diagram of spectrograms 1200 of an example embodiment of the invention which relates to neurological examination algorithms for patients with cervical dystonia. In an embodiment, muscle groups involved in cervical dystonia in this case were analyzed and the test demonstrates hyperactivity and increased tone in the right trapezius (e.g., R Trap 1210) compared to the left (e.g., L Trap 1205), left sternocleidomastoid muscle (SCM) (e.g., L SCM 1215) compared to the right (e.g., R SCM 1220), right splenius (e.g., R Splenius 1225) compared to the left (e.g., L Splenius 1230), and left levator scapula muscle (e.g., L Splenius 1235) compared to the right (e.g., R Splenius 1240). The result of the test reveals the diagnosis of cervical dystonia and the type of impairment, which is right torticollis, right laterocollis according to an example embodiment of the present invention.

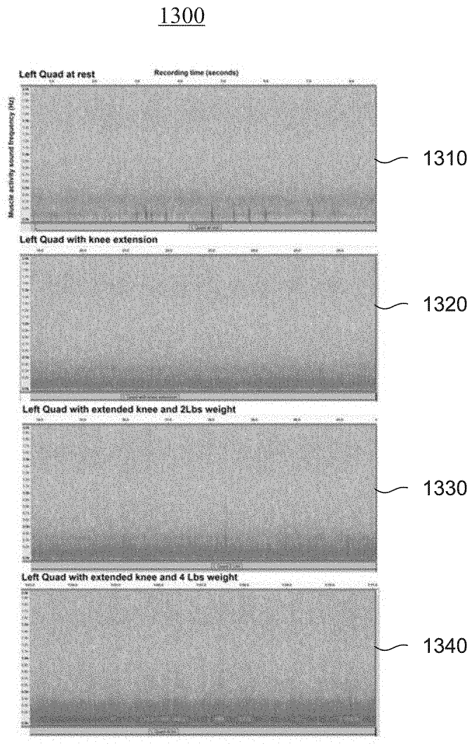

FIG. 13 depicts spectrograms 1300 of muscle strength examination, in accordance with an example embodiment of the present invention.

FIG. 13 illustrates spectrograms 1300 of examination of muscle strength and neuromuscular fitness by evaluating neuromuscular unit recruitment, comparing muscle tone at rest (e.g., L quad 1310), with muscle tone in muscle extension (e.g., L quad knee extension 1320), and while holding weights (e.g., L quad knee extension and 2 lbs. load 1330, L quad knee extension and 4 lbs. load 1340), according to an example embodiment of the present invention.

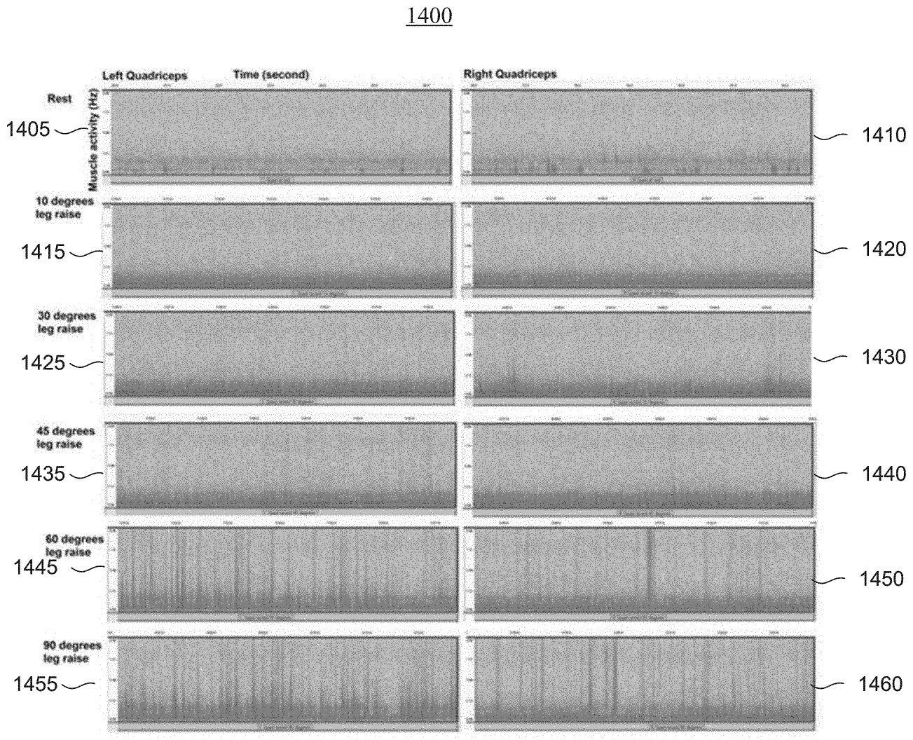

FIG. 14 depicts spectrograms 1400 of motor examination of muscle strength activity, in accordance with an example embodiment of the present invention.

FIG. 14 illustrates spectrograms 1400 of motor examination, including muscle tone at rest (e.g., L quad 1405, R quad 1410), moving extremities against gravity at different degrees (e.g., 10° with L quad leg raise 1415 and R quad leg raise 1420, 30° with L quad leg raise 1425 and R quad leg raise 1430, 45° with L quad leg raise 1435 and R quad leg raise 1440, 60° with L quad leg raise 1445 and R quad leg raise 1450, 90° with L quad leg raise 1455 and R quad leg raise 1460), with real-time muscle activity recording, as a criterion for muscle strength measurement, or neuromuscular fitness, or abnormal movements screening, including tremor or ataxia.

FIG. 15 depicts a block diagram of a computing device 1500 of distributed computing environment, in accordance with an embodiment of the present invention. For example, FIG. 15 depicts a block diagram of computing device 1500 suitable for server(s) 125 and computing device 120, in accordance with an illustrative embodiment of the present invention. It should be appreciated that FIG. 15 provides only an illustration of one implementation and does not imply any limitations with regard to the environments in which different embodiments may be implemented. Many modifications to the depicted environment may be made.

Computing device 1500 includes communications fabric 1502, which provides communications between cache 1516, memory 1506, persistent storage 1508, communications unit 1510, and input/output (I/O) interface(s) 1512. Communications fabric 1502 can be implemented with any architecture designed for passing data and/or control information between processors (such as microprocessors, communications and network processors, etc.), system memory, peripheral devices, and any other hardware components within a system. For example, communications fabric 1502 can be implemented with one or more buses or a crossbar switch.

Memory 1506 and persistent storage 1508 are computer readable storage media. In this embodiment, memory 1506 includes random access memory (RAM). In general, memory 1506 can include any suitable volatile or non-volatile computer readable storage media. Cache 1516 is a fast memory that enhances the performance of computer processor(s) 1504 by holding recently accessed data, and data near accessed data, from memory 1506.

Programs may be stored in persistent storage 1508 and in memory 1506 for execution and/or access by one or more of the respective computer processors 1504 via cache 1516. In an embodiment, persistent storage 1508 includes a magnetic hard disk drive. Alternatively, or in addition to a magnetic hard disk drive, persistent storage 1508 can include a solid-state hard drive, a semiconductor storage device, read-only memory (ROM), erasable programmable read-only memory (EPROM), flash memory, or any other computer readable storage media that is capable of storing program instructions or digital information.

The media used by persistent storage 1508 may also be removable. For example, a removable hard drive may be used for persistent storage 1508. Other examples include optical and magnetic disks, thumb drives, and smart cards that are inserted into a drive for transfer onto another computer readable storage medium that is also part of persistent storage 1508.

Communications unit 1510, in these examples, provides for communications with other data processing systems or devices. In these examples, communications unit 1510 includes one or more network interface cards. Communications unit 1510 may provide communications through the use of either or both physical and wireless communications links. Programs, as described herein, may be downloaded to persistent storage 1508 through communications unit 1510.

I/O interface(s) 1512 allows for input and output of data with other devices that may be connected to computing device 120. For example, I/O interface 1512 may provide a connection to external devices 1518 such as image sensor 130, a keyboard, a keypad, a touch screen, and/or some other suitable input device. External devices 1518 can also include portable computer readable storage media such as, for example, thumb drives, portable optical or magnetic disks, and memory cards. Software and data 1514 used to practice embodiments of the present invention can be stored on such portable computer readable storage media and can be loaded onto persistent storage 1508 via I/O interface(s) 1512. I/O interface(s) 1512 also connect to a display 1520.

Display 1520 provides a mechanism to display data to a user and may be, for example, a computer monitor.

Software and data 1514 described herein is identified based upon the application for which it is implemented in a specific embodiment of the invention. However, it should be appreciated that any particular program nomenclature herein is used merely for convenience, and thus the invention should not be limited to use solely in any specific application identified and/or implied by such nomenclature.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the invention. The terminology used herein was chosen to best explain the principles of the embodiment, the practical application or technical improvement over technologies found in the marketplace, or to enable others of ordinary skill in the art to understand the embodiments disclosed herein.

What is claimed is:

1. A computer-implemented method, comprising:
receiving, by one or more processors, biometric activity data corresponding to user extremity movement from a mobile device associated with a user;
transmitting, by one or more processors, the biometric activity data to a machine learning model;
processing, by one or more processors, using the machine learning model, the biometric activity data to generate first model output data corresponding to a first score; and
determining, by one or more processors, that the first model output data corresponds to a neurological disorder classification based at least on the first score exceeding a predetermined threshold.

2. The computer-implemented method of claim 1, further comprising:
presenting, by one or more processors, instructions via a user interface of the mobile device instructing the user to perform one or more tasks; and
responsive to presenting the instructions, receiving, by one or more processors, audio data from a biometric sensor of the mobile device contemporaneous with the one or more tasks.

3. The computer-implemented method of claim 2, wherein the biometric activity data is based at least on the audio data received contemporaneously with the user performing the one or more tasks.

4. The computer-implemented method of claim 2, wherein the one or more tasks comprise positioning the biometric sensor of the mobile device at a specific location about the body of the user to capture the audio data while the user is at rest or performing the one or more tasks.

5. The computer-implemented method of claim 4, wherein the specific location may be selected from a group consisting of left upper extremity, right upper extremity, left lower extremity, right lower extremity, left hand, right hand, left foot, and right foot.

6. The computer-implemented method of claim 1, further comprising:
receiving, by one or more processors, at the machine learning model, training data corresponding to normal neurological disorder profiles and abnormal neurological disorder profiles; and
processing, by one or more processors, at the machine learning model, the training data to configure a trained machine learning model.

7. The computer-implemented method of claim 6, further comprising:
transmitting, by one or more processors, the biometric activity data to the trained machine learning model;
processing, by one or more processors, using the trained machine learning model, the biometric activity data to generate second model output data corresponding to a second score.

8. A computer program product, comprising:
one or more computer readable storage media and program instructions stored on the one or more computer readable storage media, the program instructions comprising:
program instructions to receive biometric activity data corresponding to user extremity movement from a mobile device associated with a user;
program instructions to transmit the biometric activity data to a machine learning model;
program instructions to process using the machine learning model, the biometric activity data to generate first model output data corresponding to a first score;
program instructions to determine that the first model output data corresponds to a neurological disorder classification based at least on the first score exceeding a predetermined threshold.

9. The computer program product of claim 8, further comprising:
program instructions to present instructions via a user interface of the mobile device instructing the user to perform one or more tasks; and
responsive to the program instructions to present the instructions, program instructions to receive audio data from a biometric sensor of the mobile device contemporaneous with the one or more tasks.

10. The computer program product of claim 9, wherein the biometric activity data is based at least on receiving the audio data contemporaneously with the one or more tasks.

11. The computer program product of claim 9, wherein the one or more tasks comprise positioning the biometric sensor of the mobile device at a specific location about the body of the user to capture the audio data while the user is at rest or performing the one or more tasks.

12. The computer program product of claim 11, wherein the specific location may be selected from a group consisting of left upper extremity, right upper extremity, left lower extremity, right lower extremity, left hand, right hand, left foot, and right foot.

13. The computer program product of claim 8, further comprising:
program instructions to receive at the machine learning model, training data corresponding to normal neurological disorder profiles and abnormal neurological disorder profiles; and
program instructions to process at the machine learning model, the training data to configure a trained machine learning model.

14. The computer program product of claim 13, further comprising:
program instructions to transmit the biometric activity data to the trained machine learning model; and
program instructions to process using the trained machine learning model, the biometric activity data to generate second model output data corresponding to a second score.

15. A computer system, comprising:
one or more computer processors;
one or more computer readable storage media;
program instructions stored on the one or more computer readable storage media for execution by at least one of the one or more processors, the program instructions comprising:
program instructions to receive biometric activity data corresponding to user extremity movement from a mobile device associated with a user;
program instructions to transmit the biometric activity data to a machine learning model;
program instructions to process using the machine learning model, the biometric activity data to generate first model output data corresponding to a first score;
program instructions to determine that the first model output data corresponds to a neurological disorder classification based at least on the first score exceeding a predetermined threshold.

16. The computer system of claim 15, further comprising:
program instructions to present instructions via a user interface of the mobile device instructing the user to perform one or more tasks; and
responsive to the program instructions to present the instructions, program instructions to receive audio data from a biometric sensor of the mobile device contemporaneous with the one or more tasks.

17. The computer system of claim 16, wherein the biometric activity data is based at least on receiving the audio data contemporaneously with the one or more tasks.

18. The computer system of claim 16, wherein the one or more tasks comprise positioning the biometric sensor of the mobile device at a specific location about the body of the user to capture the audio data while the user is at rest or performing the one or more tasks.

19. The computer system of claim 18, wherein the specific location may be selected from a group consisting of left upper extremity, right upper extremity, left lower extremity, right lower extremity, left hand, right hand, left foot, and right foot.

20. The computer system of claim 15, further comprising:
program instructions to receive at the machine learning model, training data corresponding to normal neurological disorder profiles and abnormal neurological disorder profiles;
program instructions to process at the machine learning model, the training data to configure a trained machine learning model;
program instructions to transmit the biometric activity data to the trained machine learning model; and
program instructions to process using the trained machine learning model, the biometric activity data to generate second model output data corresponding to a second score.
